(19) United States
(12) Patent Application Publication
(10) Pub. No.: US 2025/0186506 A1
(43) Pub. Date: Jun. 12, 2025

(54) HUMAN UMBILICAL CORD COMPOSITION FOR TREATMENT OF PEYRONIE'S DISEASE

(71) Applicant: BioStem Technologies, Inc., Pompano Beach, FL (US)

(72) Inventors: Wendy W. Weston, Coral Springs, FL (US); Jason V Matuszewski, Boca Raton, FL (US); Taylor J Sabol, Pompano Beach, FL (US); Grace Sangeun Tran, Boca Raton, FL (US)

(21) Appl. No.: 19/056,248

(22) Filed: Feb. 18, 2025

Related U.S. Application Data
(63) Continuation-in-part of application No. 18/130,555, filed on Apr. 4, 2023.
(60) Provisional application No. 63/438,429, filed on Jan. 11, 2023.

(51) Int. Cl. A61K 35/51; A61K 9/00; A61K 38/17; A61K 38/18; A61K 38/20; A61K 38/39; A61K 38/57

(52) U.S. Cl. CPC A61K 35/51; A61K 9/0019; A61K 38/1709; A61K 38/1833; A61K 38/2006; A61K 38/39; A61K 38/57

(57) ABSTRACT

A processed human umbilical cord composition for the treatment of Peyronie's disease by intracorporeal injection in a subject in need thereof with an effective amount of the composition. The composition including an aqueous human umbilical cord filtrate having endogenous hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), transthyretin, tissue inhibitor of metalloproteinase 1 (TIMP-1), aggrecan, or a combination thereof therein at effective amount to reduce size of a Peyronie's disease plaque.

### TECHNICAL FIELD

The present invention relates generally to the field of umbilical cord derived compositions, and more particularly, to a non-immunogenic composition(s) derived from human umbilical cords used for treatment of Peyronie's disease.

### BACKGROUND

The human penis is an externally located component of the male reproductive system consisting of three chambers made up of sponge-like tissue surrounded by the tunica albuginea.

During sexual arousal, a physiological response occurs in which the penile tissue fills with blood perfused from elsewhere in the human body thereby increasing penis rigidity. Several penile disorders including erectile dysfunction (ED) and Peyronie's disease (PD) affect and/or hinder this physiological response. ED is generally defined as the inability to obtain and/or maintain an erection for sexual intercourse. It is a common condition, affecting about half of American men over 40. Causes of ED include vascular disorders, nerve disorders, psychological stresses, penile injury, chronic illness and unhealthy lifestyle habits.

In addition to the above-mentioned causes of ED, ED sometimes results from Peyronie's disease. Peyronie's disease is the development of plaques (i.e., scar tissue) inside the tunica albuginea of the penis. When these plaques are high in number and/or cover a large surface area within the penis, curved, painful erections results, which often leads to ED making sexual intercourse very painful, difficult, and/or impossible.

Approximately 1 in 100 men in the United States over the age of 18 have been diagnosed with Peyronie's disease and the chance of developing Peyronie's disease increases with age. A significant number of men with PD develop erectile dysfunction. The exact cause of PD is unknown but is theorized to result from injury.

Scarring (plaques) may develop in the elastic layers that surround the erectile tissue reducing the elasticity of the penis in the area affected. Symptoms of PD include scar tissue that can be felt under the skin of the penis, a significant bend or curve of the penis, difficulty getting or keeping an erection, pain in the penis, and shortening of the penis. Surgical treatments include removing the plaque, shortening the tissue on the side opposite the plaque to even out the bend and penile implant. These procedures risk loss of erectile function or permanent shortening of the penis.

Non-surgical treatments utilize injections of various compositions to soften the plaques and correct the curvature. These compositions currently include XIAFLEX®, which is composed of collagenase derived from bacteria for the purpose of dissolving the plaque collagens. XIAFLEX® carries risk of corporal rupture, penile hematoma, ED and blood in the urine. Verapamil is a calcium channel inhibitor used to stop the progression of plaque formation. Side effects include dizziness, weakness, nausea and sweating. Verapamil also has a long list of drug interactions. Steroid injections have been used for many years. Many patients find the injections painful. The side effects of corticosteroids are reduction in immune system function, infection at the injection site, reduction in the size of penile tissue, thinning of the skin and complication of any future surgical procedures. Radiation therapy must be used in the early stages of PD to be effective and is used to stop the progression of PD. Studies for this approach lack solid controls and exposure of reproductive tissue to radiation can be a deterrent for many patients.

### SUMMARY

In view of the above problems existing with current Peyronie's disease treatments, a need exists for the treatment of penile defects (e.g., Peyronie's disease) that exhibit minimal side effects while achieving resolution of the penile defect. The compositions and methods disclosed herein achieve this objective by utilizing a processed human umbilical cord composition used as an intracorporeal injectable treatment for Peyronie's disease in subjects in need thereof (e.g., human subjects in need thereof). Moreover, it is an object of the invention to provide compositions derived from human umbilical cord(s) that mimic, include, and/or retain a cellular and/or extracellular profile similar to the endogenous profile of a human umbilical cord (e.g., in vivo), especially when compared with various previously mentioned umbilical cord isolates. These compositions are prepared with fresh human umbilical cord (harvested and processed within 48 to 72 hours of extraction from the human subject) and, unlike the prior art compositions, are advantageously not subjected to biochemical and/or enzymatic digestion, which results in the compositions including and/or retaining a significant proportion of the cellular and/or extracellular profile (of a human umbilical cord in vivo).

In certain aspects, disclosed is a processed human umbilical cord composition for the treatment of Peyronie's disease by intracorporeal injection in a subject in need thereof with an effective amount of the composition, the composition includes an aqueous human umbilical cord filtrate having endogenous hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), transthyretin, tissue inhibitor of metalloproteinase 1 (TIMP-1), aggrecan, or a combination thereof therein at effective amount to reduce size of a Peyronie's disease plaque. In certain aspects, the aqueous human umbilical cord filtrate is a solution in which no settling, separation, and/or precipitation is observed after one month, two months, three months, four months, five months, six months, or more while being stored. In certain aspects and when preparing the composition, no exogenous enzymes are introduced therein, which avoids exogenous enzymatic degradation/digestion.

In certain aspects, the aqueous human umbilical cord filtrate comprises acellular Wharton's jelly, hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), transthyretin, tissue inhibitor of metalloproteinase 1 (TIMP-1), aggrecan, or a combination thereof. In certain aspects, the aqueous human umbilical cord filtrate comprises acellular Wharton's jelly, hyaluronan (HA) at a concentration ranging from 3.0×10⁶ pg/mL to 4.0×10⁸ pg/mL, more preferably from 4.5×10⁶ pg/mL to 3.15×10⁸ pg/mL, insulin growth factor binding protein-1 (IGFBP-1) at a concentration ranging from 2.0×10³ pg/mL to 6.0×10⁶ pg/mL, more preferably from 2.25×10³ pg/mL to 4.7×10⁶ pg/mL, sulfated glycosaminoglycans (sGAGs) at a concentration ranging from 2.0×10⁷ pg/mL to 3.0×10⁹ pg/mL, more preferably from 2.8×10⁷ pg/mL to 2.3×10⁹ pg/mL, interleukin-1 receptor antagonist (IL-1ra) at a concentration ranging from 2.0×10² pg/mL to 4.0×10⁴ pg/mL, more preferably from 3.75×10² pg/mL to 3.0×10⁴ pg/mL, transthyretin at a concentration ranging from 1.75×10² pg/mL to 6.0×10⁵ pg/mL, more preferably from 1.8×10² pg/mL to 5.3×10⁴ pg/mL, tissue inhibitor of metalloproteinase 1 (TIMP-1) at a concentration ranging from 3.0×10³ pg/mL to 8.0×10⁵ pg/mL, more preferably from 4.0×10³ pg/mL to 7.75×10⁵ pg/mL, aggrecan at a concentration ranging from 0 pg/mL to 8.0×10⁴ pg/mL, more preferably from 1×10¹ pg/mL to 7.5×10⁴ pg/mL or a combination thereof.

In certain aspects, the aqueous human umbilical cord filtrate further includes an isotonic solution, which may be included in the disclosed compositions as a carrier and/or as a diluent.

In certain aspects, the isotonic solution is phosphate buffered saline (1×PBS), lactated ringers (NaCl 6 g/L, Sodium Lactate 3.1 g/L, KCl 0.3 g/L, and CaCl 0.2 g/L at pH 6.5), isotonic saline (0.9 wt % NaCl), Plasma-Lyte® (NaCl 5.26 g/L, KCl 0.37 g/L, Magnesium Chloride hexahydrate 0.30 g/L, Sodium Acetate trihydrate 3.68 g/L, Sodium Gluconate 5.02 g/L at pH 7.4).

In certain aspects, the aqueous human umbilical cord filtrate comprises particles from a human umbilical cord tissue therein that are less than 100 μm in diameter therein and that are preferably less than 50 μm in diameter, more preferably less than 25 μm in diameter, even more preferably less than 10 μm in diameter.

In certain aspects, the aqueous human umbilical cord filtrate is sterile.

In certain aspects, the aqueous human umbilical cord filtrate are non-immunogenic.

Also disclosed are kits comprising the processed human umbilical cord composition for the treatment of Peyronie's disease by intracorporeal injection in a subject in need thereof. In certain aspects, the composition is pre-packaged in a vial (sterile vial), ampule (sterile ampule), or a pre-loaded syringe (sterile pre-loaded syringe) preferably having a predetermined volume and concentration of, for example, endogenous hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), transthyretin, tissue inhibitor of metalloproteinase 1 (TIMP-1), aggrecan, or a combination thereof to reduce size of a Peyronie's disease plaque. In certain aspects, the predetermined volume includes 0.25 mL to 20.0 mL, preferably 0.5 mL to 10.0 mL, and most preferably 1.0 to 5.0 mL and may be used for 1 to 10 treatments (injections) with the composition to treat Peyronie's disease plaques or as determined by a physician. Also in certain aspects, the aqueous human umbilical cord filtrate comprises acellular Wharton's jelly, hyaluronan (HA) at a concentration ranging from 3.0×10⁶ pg/mL to 4.0×10⁸ pg/mL, more preferably from 4.5×10⁶ pg/mL to 3.15×10⁸ pg/mL, insulin growth factor binding protein-1 (IGFBP-1) at a concentration ranging from 2.0×10³ pg/mL to 6.0×10⁶ pg/mL, more preferably from 2.25×10³ pg/mL to 4.7×10⁶ pg/mL, sulfated glycosaminoglycans (sGAGs) at a concentration ranging from 2.0×10⁷ pg/mL to 3.0×10⁹ pg/mL, more preferably from 2.8×10⁷ pg/mL to 2.3×10⁹ pg/mL, interleukin-1 receptor antagonist (IL-1ra) at a concentration ranging from 2.0×10² pg/mL to 4.0×10⁴ pg/mL, more preferably from 3.75×10² pg/mL to 3.0×10⁴ pg/mL, transthyretin at a concentration ranging from 1.75×10² pg/mL to 6.0×10⁵ pg/mL, more preferably from 1.8×10² pg/mL to 5.3×10⁴ pg/mL, pg/mL tissue inhibitor of metalloproteinase 1 (TIMP-1) at a concentration ranging from 3.0×10³ pg/mL to 8.0×10⁵ pg/mL, more preferably from 4.0×10³ pg/mL to 7.75×10⁵ pg/mL, aggrecan at a concentration ranging from 0 pg/mL to 8.0×10⁴ pg/mL, more preferably from 1×10¹ pg/mL to 7.5×10⁴ pg/mL or a combination thereof.

In certain aspects, the aqueous human umbilical cord filtrate is preferably not subjected to exogenous enzymatic digestion.

In certain aspects, the aqueous human umbilical cord filtrate of the kit further comprises an isotonic solution.

In certain aspects, the isotonic solution of the kit is phosphate buffered saline (1×PBS), lactated ringers (NaCl 6 g/L, Sodium Lactate 3.1 g/L, KCl 0.3 g/L, and CaCl 0.2 g/L at pH 6.5), isotonic saline (0.9 wt % NaCl), Plasma-Lyte® (NaCl 5.26 g/L, KCl 0.37 g/L, Magnesium Chloride hexahydrate 0.30 g/L, Sodium Acetate trihydrate 3.68 g/L, Sodium Gluconate 5.02 g/L at pH 7.4).

In certain aspects, the aqueous human umbilical cord filtrate of the kit comprises particles from a human umbilical cord tissue that are less than 100 μm in diameter therein and that are preferably less than 50 μm in diameter, more preferably less than 25 μm in diameter, even more preferably less than 10 μm in diameter. In certain aspects, the aqueous human umbilical cord filtrate is a solution in which no settling, separation, and/or precipitation is observed after one month, two months, three months, four months, five months, six months, or more while being stored.

In certain aspects, the aqueous human umbilical cord filtrate of the kit are sterile.

In certain aspects, the aqueous human umbilical cord filtrate of the kit are non-immunogenic.

In certain aspects, also disclosed is a method for reducing size of a Peyronie's disease plaque in a human subject in need thereof comprising contacting the Peyronie's disease plaque with a predetermined volume of the above-disclosed composition. In certain aspects, the contacting step includes an intracorporeal injection of the above-disclosed composition at a predetermined volume having a predetermined concentration of, for example, endogenous hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), transthyretin, tissue inhibitor of metalloproteinase 1 (TIMP-1), aggrecan, or a combination thereof at a predetermined frequency in a subject (human subject) in need thereof to reduce plaque size. In certain aspects, the above is repeated at predetermined frequencies to further reduce plaque size and/or to maintain plaque reduction in the subject.

In additional aspects, also disclosed is a method of making the processed human umbilical cord composition for the treatment of Peyronie's disease by intracorporeal injection in a subject in need there, the method comprising: (a) providing a human umbilical cord; (b) washing the human umbilical cord with an isotonic solution; (c) grinding the washed human umbilical cord of step (b) thereby forming ground human umbilical cord tissue; (d) separating the ground human umbilical cord tissue of step (c) into a solid retentate and an aqueous human umbilical cord supernatant; optionally further processing the solid retentate of step (d) into a micronized human umbilical cord composition; and (e) filtering and diluting the aqueous human umbilical cord supernatant thereby forming an aqueous human umbilical cord filtrate, and if present, the aqueous human umbilical cord filtrate having particles from the ground human umbilical cord tissue that are less than 100 μm in diameter therein. In this aspect, none of steps (a)-(e) include introduction of exogenous enzymes resulting in exogenous enzymatic degradation/digestion.

In certain aspects the human umbilical cord of the method is obtained from a subject and is subsequently subjected to steps (a)-(c) within 48 to 96 hours, more preferably within 48 to 72 hours post-childbirth and/or caesarean section and/or human umbilical cord extraction event.

In certain aspects, the method further comprises, between steps (a)-(c), removing any blood clots present within the human umbilical cord.

In certain aspects, step (b) is repeated between one to five times by discarding the used isotonic solution and providing new isotonic solution and again washing the human umbilical cord with the new isotonic solution.

In certain aspects, 15 to 80 grams of human umbilical cord is provided in step (a) and is subsequently subjected to steps (b)-(f).

In certain aspects, the isotonic solution is phosphate buffered saline (or one of lactated ringers (NaCl 6 g/L, Sodium Lactate 3.1 g/L, KCl 0.3 g/L, and CaCl 0.2 g/L at pH 6.5), isotonic saline (0.9 wt % NaCl), Plasma-Lyte® (NaCl 5.26 g/L, KCl 0.37 g/L, Magnesium Chloride hexahydrate 0.30 g/L, Sodium Acetate trihydrate 3.68 g/L, Sodium Gluconate 5.02 g/L at pH 7.4)) provided at a volume ranging from 300 mL to 1000 mL per washing step (b).

In certain aspects, a grinding tool configured to grind and/or mince the washed human umbilical cord is used during step (c) and grinds the washed human umbilical cord at a range of 40 to 200 revolutions per minute (RPM) until the umbilical cord has been fully ground.

In certain aspects, the method includes before step (d) contacting the ground umbilical cord tissue formed in step (c) with a filter and subsequently filtering the ground umbilical cord tissue to form the solid retentate retained on the filter and the aqueous human umbilical cord supernatant of step (d) that has passed through the filter. In this aspect, the filter has a porosity ranging from 100 μm to 200 μm.

In certain aspects, the further processing step of the solid retentate is present and is a milling, freeze drying, or dehydration process that forms the micronized human umbilical cord composition of step (e) having particle sizes ranging from greater than 1 μm to less than 300 μm and more preferably particle sizes ranging from greater than 1 μm to 100 μm and even more preferably from greater than 1 μm to 50 μm and even more preferably from greater than 1 μm to 25 μm. Moreover, the particles of the micronized human umbilical cord are polydisperse.

In certain aspects, the further processing step of the solid retentate is present and is a cryomilling process in which the solid retentate of step (d) is placed into a liquid nitrogen cooled cryomill chamber and subjected to grinding therein thereby forming the micronized human umbilical cord composition having particle sizes ranging from greater than 1 μm to less than 300 μm and more preferably particle sizes ranging from greater than 1 μm to 100 μm, and even more preferably from greater than 1 μm to 50 μm and even more preferably from greater than 1 μm to 25 μm. Moreover, the particles of the micronized human umbilical cord are polydisperse.

In certain aspects, step (e) comprises a plurality of filtration steps comprising: (i) filtering the aqueous human umbilical cord supernatant through a first filter having a porosity ranging from 30 μm to 40 μm thereby forming a second human umbilical cord supernatant; (ii) filtering the second human umbilical cord supernatant through a second filter having a porosity ranging from 12.5 μm to 25 μm thereby forming a third human umbilical cord supernatant; and (iii) filtering the third human umbilical cord supernatant through a third filter having a porosity ranging from 4 μm to 10 μm thereby forming the aqueous human umbilical cord filtrate. Thus, in certain aspects, the second human umbilical cord supernatant would include particles from the human umbilical cord tissue that are less than 40 μm; the third human umbilical cord supernatant would include particles from the human umbilical cord tissue that are less than 25 μm; and the aqueous human umbilical cord filtrate would include particles from the human umbilical cord tissue that are less than 10 μm. In certain aspects, the aqueous human umbilical cord filtrate is a solution in which no settling, separation, and/or precipitation is observed after one month, two months, three months, four months, five months, six months, or more while being stored.

In certain aspects, the micronized human umbilical cord composition of the method comprises collagen, fibronectin, IGFBP-1, sGAGs, hyaluronan, or any combination thereof.

In certain aspects, the micronized human umbilical cord of the method composition is a dried micronized human umbilical cord tissue having a particle diameter size ranging from greater than 1 μm to 300 μm and comprising collagen, fibronectin, IGFBP-1, sGAGs, hyaluronan, or any combination thereof.

In certain aspects, the aqueous human umbilical cord filtrate of the method comprises acellular Wharton's jelly, hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), transthyretin, tissue inhibitor of metalloproteinase 1 (TIMP-1), aggrecan, or a combination thereof. In certain aspects, the aqueous human umbilical cord filtrate comprises acellular Wharton's jelly, hyaluronan (HA) at a concentration ranging from 3.0×10⁶ pg/mL to 4.0×10⁸ pg/mL, more preferably from 4.5×10⁶ pg/mL to 3.15×10⁸ pg/mL, insulin growth factor binding protein-1 (IGFBP-1) at a concentration ranging from 2.0×10³ pg/mL to 6.0×10⁶ pg/mL, more preferably from 2.25×10³ pg/mL to 4.7×10⁶ pg/mL, sulfated glycosaminoglycans (sGAGs) at a concentration ranging from 2.0×10⁷ pg/mL to 3.0×10⁹ pg/mL, more preferably from 2.8×10⁷ pg/mL to 2.3×10⁹ pg/mL, interleukin-1 receptor antagonist (IL-1ra) at a concentration ranging from 2.0×10² pg/mL to 4.0×10⁴ pg/mL, more preferably from 3.75×10² pg/mL to 3.0×10⁴ pg/mL, transthyretin at a concentration ranging from 1.75×10² pg/mL to 6.0×10⁵ pg/mL, more preferably from 1.8×10² pg/mL to 5.3×10⁴ pg/mL, tissue inhibitor of metalloproteinase 1 (TIMP-1) at a concentration ranging from 3.0×10³ pg/mL to 8.0×10⁵ pg/mL, more preferably from 4.0×10³ pg/mL to 7.75×10⁵ pg/mL, aggrecan at a concentration ranging from 0 pg/mL to 8.0×10⁴ pg/mL, more preferably from 1×10¹ pg/mL to 7.5×10⁴ pg/mL or a combination thereof.

In certain aspects, both the micronized human umbilical cord composition and the aqueous human umbilical cord filtrate of the method are sterile.

In certain aspects, both the micronized human umbilical cord composition and the aqueous human umbilical cord filtrate of the method are non-immunogenic.

In certain aspects, the method further comprises (f) placing and sealing aqueous human umbilical cord filtrate composition in a sterile container for subsequent use. For example, the aqueous human umbilical cord filtrate is pre-packaged in a vial (sterile vial), ampule (sterile ampule), or a pre-loaded syringe (sterile pre-loaded syringe) preferably having a predetermined volume and concentration of, for example, endogenous hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), transthyretin, tissue inhibitor of metalloproteinase 1 (TIMP-1), aggrecan, or a combination thereof to reduce size of a Peyronie's disease plaque. In certain aspects, the predetermined volume includes 0.25 mL to 20.0 mL, preferably 0.5 mL to 10.0 mL, and most preferably 1.0 to 5.0 mL and may be used for 1 to 10 treatments (injections) with the composition to treat Peyronie's disease plaques or as determined by a physician

Embodiments of the invention can include one or more or any combination of the above features and configurations.

Additional features, aspects and advantages of the invention will be set forth in the detailed description, which follows, and in part will be readily apparent to those skilled in the art from that description or recognized by practicing the invention as described herein. It is to be understood that both the foregoing general description and the following detailed description present various embodiments of the invention and are intended to provide an overview or framework for understanding the nature and character of the invention as it is claimed. The accompanying drawings are included to provide a further understanding of the invention and are incorporated in and constitute a part of this specification.

### BRIEF DESCRIPTION OF THE DRAWINGS

These and other features, aspects and advantages of the present invention are better understood when the following detailed description of the invention is read with reference to the accompanying drawings, in which:

FIG. 1 is a schematic depiction of the steps included for making the disclosed compositions; and

FIG. 2 are graphs showing the concentration profiles of hyaluronic acid (HA) and/or hyaluronan, fibronectin, insulin growth factor binding protein-1 (IGFBP-1), sulfated glycosaminoglycans (sGAGs), interleukin-1 receptor antagonist (IL-1ra), hepatocyte growth factor (HGF), transthyretin, tissue inhibitor of metalloproteinase 1 (TIMP-1), and aggrecan in the aqueous human umbilical cord filtrate.

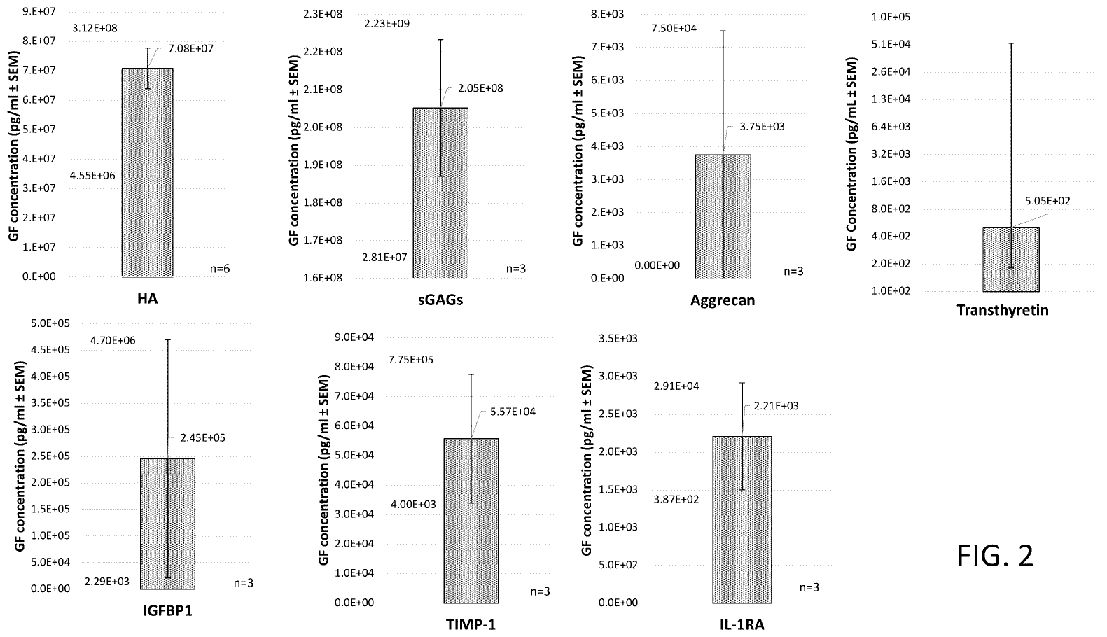

### DETAILED DESCRIPTION

The present invention will now be described more fully hereinafter with reference to the accompanying drawings in which exemplary embodiments of the invention are shown. However, the invention may be embodied in many different forms and should not be construed as limited to the representative embodiments set forth herein. The exemplary embodiments are provided so that this disclosure will be both thorough and complete and will fully convey the scope of the invention and enable one of ordinary skill in the art to make, use and practice the invention. Like reference numbers refer to like elements throughout the various drawings. Moreover, in this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within the ranges as if each numerical value and sub-range is explicitly recited. As an illustration, a numerical range of “about 1 to 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc. as well as 1, 2, 3, 4, and 5, individually. The same principle applies to ranges reciting only one numerical value as a minimum or a maximum. Furthermore, such an interpretation should apply regardless of the breadth of the range or the characteristics being described.

The compositions and methods described herein can comprise, consist of, or consist essentially of the essential elements and limitations described herein, as well as any additional or optional ingredients, components, or limitations described herein.

### Human Umbilical Cord Compositions

Disclosed herein are compositions derived from human umbilical cord(s) that mimic, include, and/or retain a cellular and/or extracellular profile similar to the endogenous profile of a human umbilical cord, for example, in vivo, especially when compared with various conventional umbilical cord isolate compositions. The disclosed compositions are prepared with fresh human umbilical cord (harvested and processed within 48 to 72 hours of extraction from the human subject) and, unlike compositions in the prior art, are advantageously not subjected to biochemical and/or enzymatic digestion, which results in the compositions including and/or retaining a significant portion of the cellular and/or extracellular profile (when compared to the endogenous profile of a human umbilical cord in vivo). Moreover, these compositions may be used for the treatment of Peyronie's disease.

In certain aspects, disclosed is a processed human umbilical cord composition for the treatment of Peyronie's disease by intracorporeal injection in a subject in need there with an effective amount of the composition, the composition includes an aqueous human umbilical cord filtrate having endogenous hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), transthyretin, tissue inhibitor of metalloproteinase 1 (TIMP-1), aggrecan, or a combination thereof therein at effective amount to reduce size of a Peyronie's disease plaque. In certain aspects, the aqueous human umbilical cord filtrate is a solution in which no settling, separation, and/or precipitation is observed after one month, two months, three months, four months, five months, six months, or more while being stored. In certain aspects and when preparing composition, no exogenous enzymes are introduced therein, which avoids exogenous enzymatic degradation/digestion. The aqueous human umbilical cord filtrate and/or processed human umbilical cord composition comprises acellular Wharton's jelly, hyaluronan (HA) at a concentration ranging from 3.0×10⁶ pg/mL to 4.0×10⁸ pg/mL, more preferably from 4.5×10⁶ pg/mL to 3.15×10⁸ pg/mL, insulin growth factor binding protein-1 (IGFBP-1) at a concentration ranging from 2.0×10³ pg/mL to 6.0×10⁶ pg/mL, more preferably from 2.25×10³ pg/mL to 4.7×10⁶ pg/mL, sulfated glycosaminoglycans (sGAGs) at a concentration ranging from 2.0×10⁷ pg/mL to 3.0×10⁹ pg/mL, more preferably from 2.8×10⁷ pg/mL to 2.3×10⁹ pg/mL, interleukin-1 receptor antagonist (IL-1ra) at a concentration ranging from 2.0×10² pg/mL to 4.0×10⁴ pg/mL, more preferably from 3.75×10² pg/mL to 3.0×10⁴ pg/mL, transthyretin at a concentration ranging from 1.75×10² pg/mL to 6.0×10⁵ pg/mL, more preferably from 1.8×10² pg/mL to 5.3×10⁴ pg/mL, tissue inhibitor of metalloproteinase 1 (TIMP-1) at a concentration ranging from 3.0×10³ pg/mL to 8.0×10⁵ pg/mL, more preferably from 4.0×10³ pg/mL to 7.75×10⁵ pg/mL, aggrecan at a concentration ranging from 0 pg/mL to 8.0×10⁴ pg/mL, more preferably from 1×10¹ pg/mL to 7.5×10⁴ pg/mL, in which any endpoint within these ranges can serve as endpoints for any additional sub-range falling therein, or a combination thereof. The processed human umbilical cord composition having predetermined volumes includes ranging from 0.25 mL to 20.0 mL, preferably 0.5 mL to 10.0 mL, and most preferably 1.0 to 5.0 mL, in which any endpoint within these ranges can serve as endpoints for any additional sub-range falling therein, and may be used for 1 to 10 treatments (injections) with the composition to treat Peyronie's disease plaques or as determined by a physician. In certain aspects, the processed human umbilical cord composition is non-immunogenic, sterile, or a combination thereof.

Without wishing to be bound by theory, it is believed that hyaluronic acid, sulfated glycosaminoglycans, aggrecan, IGFBP-1, IL-1ra, tissue inhibitor of metalloproteinase 1 (TIMP-1) and transthyretin included in the disclosed processed human umbilical cord compositions at effective concentrations treat Peyronie's disease plaques by playing a role in ECM repair, matrix remodeling, and increased hydration/water retention coupled with the anti-inflammatory characteristics of one or more of the above-mentioned components, which lead to Peryonie's plaque reduction and/or resolution.

### Method of Making Human Umbilical Cord Compositions

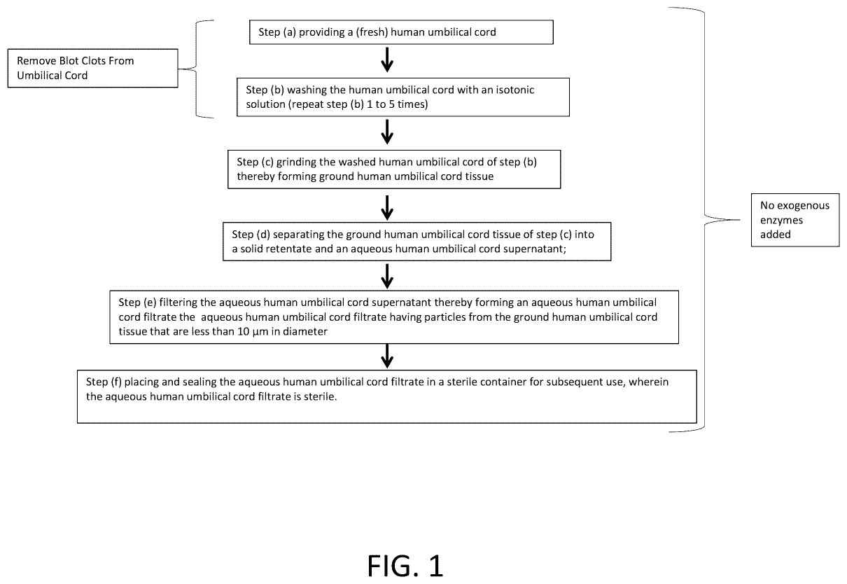

FIG. 1 provides a schematic depiction of the steps included for making the compositions disclosed herein, and as further shown in FIG. 1, none of steps include introduction of exogenous enzymes resulting in exogenous enzymatic degradation/digestion. The method of making the disclosed compositions including steps (a)-(f) discussed immediately below. Before step (a), the umbilical cord is screened for communicable diseases to ensure that the umbilical cord/umbilical cord tissue is healthy/disease free and to further minimize risk during preparation and subsequent end use of the two-part clotting compositions. After confirming that the umbilical cord/umbilical cord tissue is healthy/disease free, the umbilical cord is maintained at temperature ranging from 4° C. to 8° C. before beginning the processing of the cord in steps (a)-(f).

As shown in FIG. 1, step (a) includes providing a human umbilical cord preferably within 24 to 96 hours post-extraction from a human subject, more preferably from 24 to 72 hours post-extraction from a human subject to ensure freshness of the human umbilical cord (i.e., tissue and cells comprising the tissue) and to minimize degradation associated (enzymatic degradation) resulting from necroptosis and/or apoptosis. In this step and in order for appropriate grinding/mincing to occur (in subsequent step (c)), it is preferred that 15 to 80 gram portions and more preferably 30 to 60 gram portions of the human umbilical cord are subjected to the below mentioned method, with the average portions being approximately 40 grams.

After completing step (a), step (b) occurs. Step (b) includes placing one or more umbilical cord portions (15 to 80 gram) into a container having a predetermined volume (e.g., 300 mL to 1000 mL, preferably 500 mL) of isotonic solution in which the isotonic solution is preferably phosphate buffered saline (PBS) (i.e., 1×PBS) (or alternatively one of lactated ringers (NaCl 6 g/L, Sodium Lactate 3.1 g/L, KCl 0.3 g/L, and CaCl 0.2 g/L at pH 6.5), isotonic saline (0.9 wt % NaCl), Plasma-Lyte® (NaCl 5.26 g/L, KCl 0.37 g/L, Magnesium Chloride hexahydrate 0.30 g/L, Sodium Acetate trihydrate 3.68 g/L, Sodium Gluconate 5.02 g/L at pH 7.4)). Placing the container onto a stir plate and placing a stir bar within the container (containing the PBS and umbilical cord portions) therein and stirring (medium to high speed) the umbilical cord portions within the isotonic solution for 5 to 15 minutes to wash the umbilical cord portions. Next, washing step (b) is repeated one to five times by decanting the “used” isotonic solution and pouring new isotonic solution into the container at a predetermined volume (e.g., 300 mL to 1000 mL, preferably 500 mL) to again wash the one or more umbilical cord portions. Either before step (a), during step (a), before step (b), or during step (b) further determining whether any blood clots and/or blood pool(s)/pooling are present in the human umbilical cord and/or umbilical cord portions, and if so, removing these blood clots via suction or other mechanical removal means (e.g., scalpel and forceps) to further ensure that the presence of any immunogenic components (e.g., hemoglobin and/or heme associated components from the umbilical cord donor) are minimized in the resulting micronized human umbilical cord composition and the resulting aqueous human umbilical cord filtrate. During these washing steps, it is imperative to maintain an aseptic and/or sterile work environment to prevent and/or reduce introduction of any contaminants while making the disclosed compositions.

Upon concluding step (b), step (c) is performed in which the washed umbilical cord/umbilical cord portions (being within a predetermined volume (e.g., 75 mL to 125 mL, preferably 100 mL) of the isotonic solution) are transferred to a grinding and/or mincing apparatus such as those disclosed in U.S. Pat. No. D716,601 “Tissue Mincing Tool” and/or U.S. Pat. No. 8,967,512 “Systems And Methods For Processing Cells”, which are incorporated by reference herein in their entirety. The washed umbilical cord/umbilical cord portions are subsequently subjected to grinding and/or mincing by the grinding/mincing tool with the head of the grinding/mincing tool rotating at a range of 40 to 200 revolutions per minute (RPM) until the umbilical cord has been fully ground thereby forming ground human umbilical cord tissue. During this grinding/mincing step, it is imperative to maintain a sterile work environment to prevent and/or reduce introduction of any contaminants while making the micronized human umbilical cord composition and the aqueous human umbilical cord filtrate. In certain aspects, the grinding/mincing tool may be directly connected to an apparatus (i.e., a closed system environment as disclosed, for example, in U.S. Pat. No. 8,967,512) to further conduct steps (d)-(f) discussed below and to further maintain sterility and/or minimize the introduction of any contaminants while making the micronized human umbilical cord composition and the aqueous human umbilical cord filtrate. Alternatively, steps (d)-(f) maybe conducted in an open system/laboratory environment while performing each of the below mentioned steps.

Upon concluding step (c), step (d) is performed in which the ground/minced human umbilical cord tissue of step (c) is separated into a solid retentate and an aqueous human umbilical cord supernatant. This initial separation step may occur via a filtration process (either positive or negative pressure). For example, the minced/ground human umbilical cord tissue (of step (c) and included within a predetermined volume (e.g., 75 mL to 125 mL, preferably 100 mL) of the isotonic solution) may be placed directly on a filter having a desired porosity (e.g., 200 μm or 150 μm or 100 μm such as either a qualitative grade or quantitative grade mesh or net filter) and then force (either positive or negative pressure) may or may not be applied such that a solid retentate (solids having a size above 200 μm or 150 μm or 100 μm) remain on the filter while an aqueous human umbilical cord supernatant (having any solids therein that are less than (200 μm or 150 μm or 100 μm) are passed through the filter. The filtration step generally takes 15 seconds to 2 minutes. Again, it is imperative to maintain a sterile and/or aseptic work environment to prevent and/or reduce introduction of any contaminants throughout step (d).

Upon concluding step (d), an optional step is performed and the solid retentate of step (d) is further processed into a micronized human umbilical cord composition by subjecting the solid retentate to a milling, freeze drying (cryomilling), and/or dehydration (lyophilization) process configured to yield particles (polydisperse particles) having sizes ranging from greater than 1 μm to 300 μm, preferably greater than 1 μm to 100 μm, and more preferably greater than 1 μm to 50 μm and more preferably greater than to than 1 μm to 25 μm. In certain aspects, this optional step is a cryomilling process (as described, for example, US20160287749, US20170203004, and U.S. Pat. No. 10,105,398, which are each incorporated by reference in their entirety herein) in which the solid retentate of step (d) is placed into a liquid nitrogen cooled cryomill chamber and subjected to grinding therein thereby forming the micronized human umbilical cord composition having particle sizes ranging from greater than 1 μm to less than 300 μm, preferably greater than 1 μm to 100 μm, more preferably greater than 1 μm to 50 μm, and even more preferably from greater than 1 μm to 25 μm. The micronized human umbilical cord composition comprises collagen, fibronectin, hyaluronan, elastins, or any combination thereof.

Upon concluding step (d) (and the optional step if applicable), step (e) is performed on the aqueous human umbilical cord supernatant. Step (e) preferably includes a plurality of filtration steps including: (i) filtering the aqueous human umbilical cord supernatant through a first filter having a porosity ranging from 30 μm to 40 μm thereby forming a second human umbilical cord supernatant; (ii) filtering the second human umbilical cord supernatant through a second filter having a porosity ranging from 12.5 μm to 25 μm thereby forming a third human umbilical cord supernatant; and (iii) filtering the third human umbilical cord supernatant through a third filter having a porosity ranging from 4 μm to 10 μm thereby forming the aqueous human umbilical cord filtrate. The filtration step generally takes 15 seconds to 2 minutes at 1-5 psi vacuum to complete. In certain aspects, the force applied is a negative pressure (vacuum) and preferred because such negative pressure is less likely to damage the filter and lead to subsequent quality control issues with the resulting aqueous human umbilical cord filtrate disclosed herein. The aqueous human umbilical cord filtrate preferably includes acellular endogenous Wharton's jelly, endogenous hyaluronic acid (HA) and/or hyaluronan, endogenous fibronectin, endogenous insulin growth factor binding protein-1 (IGFBP-1), endogenous sulfated glycosaminoglycans (sGAGs), endogenous exosomes, endogenous interleukin-1 receptor antagonist (IL-1ra), endogenous hepatocyte growth factor (HGF), endogenous transthyretin, endogenous tissue inhibitor of metalloproteinase 1 (TIMP-1), endogenous aggrecan, or any combination thereof at effective amount to reduce size of a Peyronie's disease plaque when administered to a Peryronie's disease plaque by intracorporeal injection in a subject in need thereof. In certain aspects, the aqueous human umbilical cord filtrate comprises acellular Wharton's jelly, hyaluronan (HA) at a concentration ranging from 3.0×10⁶ pg/mL to 4.0×10⁸ pg/mL, insulin growth factor binding protein-1 (IGFBP-1) at a concentration ranging from 2.0×10³ pg/mL to 6.0×10⁶ pg/mL, sulfated glycosaminoglycans (sGAGs) at a concentration ranging from 2.0×10⁷ pg/mL to 3.0×10⁹ pg/mL, interleukin-1 receptor antagonist (IL-1ra) at a concentration ranging from 2.0×10² pg/mL to 4.0×10⁴ pg/mL, transthyretin at a concentration ranging from 1.75×10² pg/mL to 6.0×10⁵ pg/mL, tissue inhibitor of metalloproteinase 1 (TIMP-1) at a concentration ranging from 3.0×10³ pg/mL to 8.0×10⁵ pg/mL, aggrecan at a concentration ranging from 0 pg/mL to 8.0×10⁴ pg/mL, in which any endpoint within these ranges can serve as endpoints for any additional sub-range falling therein, or a combination thereof. Each filtration step generally takes 15 seconds to 2 minutes at 1-5 psi vacuum to complete. Moreover, the resulting aqueous human umbilical cord filtrate from the above-mentioned filtration steps is a solution in which no settling, separation, and/or precipitation is observed after one month, two months, three months, four months, five months, six months, or more while being stored.

In certain aspects, both the micronized human umbilical cord composition and the aqueous human umbilical cord filtrate are sterile, and both the micronized human umbilical cord composition and the aqueous human umbilical cord filtrate are non-immunogenic.

After concluding steps (e), step (g) is performed by placing and sealing the aqueous human umbilical cord filtrate (of step (e)) in a sterile container (e.g., vial, ampule, preloaded syringe) for subsequent use by intracorporeal injection in a subject in need thereof to treat Peyronie's disease.

In certain aspects, both the micronized human umbilical cord composition and the aqueous human umbilical cord filtrate are sterile and are configured to form a paste or lotion when admixed with one another. In this aspect, the micronized human umbilical cord composition may be preloaded into a sterile syringe; and the aqueous human umbilical cord filtrate may be preloaded into a sterile container or syringe that is separate from the micronized human umbilical cord composition, with each being sterilized and configured for sterilely mixing the two components together as desired to form the two-part clotting composition configured for wound packing and/or dental purposes and/or dental treatments disclosed herein

### Kits Containing Processed Human Umbilical Cord Composition for the Treatment of Peyronie's Disease

Also disclosed are kits comprising the processed human umbilical cord composition for the treatment of Peyronie's disease by intracorporeal injection in a subject in need thereof. In certain aspects, the composition is pre-packaged in a vial (sterile vial), ampule (sterile ampule), or a pre-loaded syringe (sterile pre-loaded syringe) preferably having a predetermined volume and concentration of, for example, endogenous hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), transthyretin, tissue inhibitor of metalloproteinase 1 (TIMP-1), aggrecan, or a combination thereof to reduce size of a Peyronie's disease plaque. In certain aspects, the predetermined volume includes 0.25 mL to 20.0 mL, preferably 0.5 mL to 10.0 mL, and most preferably 1.0 to 5.0 mL and may be used for 1 to 10 treatments (injections) with the composition to treat Peyronie's disease plaques or as determined by a physician with the treatment frequency ranging from every 2 to 6 weeks or as determined by the physician. Also in certain aspects, the aqueous human umbilical cord filtrate comprises acellular Wharton's jelly, hyaluronan (HA) at a concentration ranging from 3.0×10⁶ pg/mL to 4.0×10⁸ pg/mL, more preferably from 4.5×10⁶ pg/mL to 3.15×10⁸ pg/mL, insulin growth factor binding protein-1 (IGFBP-1) at a concentration ranging from 2.0×10³ pg/mL to 6.0×10⁶ pg/mL, more preferably from 2.25×10³ pg/mL to 4.7×10⁶ pg/mL, sulfated glycosaminoglycans (sGAGs) at a concentration ranging from 2.0×10⁷ pg/mL to 3.0×10⁹ pg/mL, more preferably from 2.8×10⁷ pg/mL to 2.3×10⁹ pg/mL, interleukin-1 receptor antagonist (IL-1ra) at a concentration ranging from 2.0×10² pg/mL to 4.0×10⁴ pg/mL, more preferably from 3.75×10² pg/mL to 3.0×10⁴ pg/mL, transthyretin at a concentration ranging from 1.75×10² pg/mL to 6.0×10⁵ pg/mL, more preferably from 1.8×10² pg/mL to 5.3×10⁴ pg/mL, tissue inhibitor of metalloproteinase 1 (TIMP-1) at a concentration ranging from 3.0×10³ pg/mL to 8.0×10⁵ pg/mL, more preferably from 4.0×10³ pg/mL to 7.75×10⁵ pg/mL, aggrecan at a concentration ranging from 0 pg/mL to 8.0×10⁴ pg/mL, more preferably from 1×10¹ pg/mL to 7.5×10⁴ pg/mL, in which any endpoint within these ranges can serve as endpoints for any additional sub-range falling therein, or a combination thereof.

### Methods of Use

Without wishing to be bound by theory, it is envisioned that certain compositions disclosed above in the “Method of Making Human Umbilical Cord Compositions” section may be particularly useful for wound packing, clotting, and wound healing purposes and would advantageously produce very little immunogenic response to the composition's non-immunogenic characteristics/properties.

Collagen, fibronectin and, hyaluronan found within the composition provide substrate for clotting factors to bind and signaling for cell attachment and growth. VEGFR1, HGF, interleukin antagonists (IL-1ra), bFGF and PDGF-BB provide cell growth signaling and anti-inflammatory effects thereby providing various therapeutic effects such as wound healing.

The advantageous uses may be achieved due to the ease of use (e.g., ease of preparation and ease of readily modifying the composition's viscosity when mixing the micronized human umbilical cord composition and aqueous human umbilical cord filtrate) of the compositions for the desired purpose. In certain aspects, viscosity may be modified as well by the addition of an isotonic solution such as those disclosed herein.

For example, it is envisioned that the compositions disclosed herein may be used for dental purposes (periodontic and/or endodontic purposes) and more particularly for packing a subject's gums post-tooth extraction. In this particular use, a subject's gum(s) are packed with the compositions disclosed herein almost immediately post-tooth extraction. In this aspect, the micronized human umbilical cord composition and aqueous human umbilical cord filtrate are mixed (sterilely mixed) at an effective viscosity to induce blood clotting; and the cavity within the subject's gums formed by the tooth extraction is packed with the mixed composition (sterile composition) to induce blood clotting within the packed cavity. In certain aspects, the micronized human umbilical cord composition and the aqueous human umbilical cord filtrate are both sterile and non-immunogenic and are mixed at a ratio of 2:1 to 1:2 micronized human umbilical cord composition and the aqueous human umbilical cord filtrate during this method. If very viscous mixed composition is desired, a higher proportion of the micronized human umbilical cord composition is mixed with a lower proportion the aqueous human umbilical cord filtrate, and conversely, if a less viscous mixed composition is desired, a higher proportion of the aqueous human umbilical cord filtrate is mixed with a lower proportion of the micronized human umbilical cord composition. The above-mentioned packing may be repeated, as necessary.

While the above specifically envisions dental uses, it is further envisioned that the disclosed compositions may have more general applications in the medical field such as general wound packing (occurring in surgical procedures and/or acute trauma resulting in open external and/or internal wounds) and/or wound healing. In this aspect, it is envisioned that one would initially assess the wound to generally determine the overall viscosity and thickness of the (mixed) two-part composition needed to, for example, pack and/or treat a subject's wound. Next, one sterilely mixes the composition at an effective viscosity to induce blood clotting; and then sterilely packs the subject's wound with the sterilely mixed composition to induce blood clotting within the sterilely packed wound. In certain aspects, the micronized human umbilical cord composition and the aqueous human umbilical cord filtrate are both sterile and non-immunogenic and are mixed at a ratio of 2:1 to 1:2 micronized human umbilical cord composition and the aqueous human umbilical cord filtrate during this method. If very viscous mixed composition is desired, a higher proportion of the micronized human umbilical cord composition is mixed with a lower proportion the aqueous human umbilical cord filtrate, and conversely, if a less viscous mixed composition is desired, a higher proportion of the aqueous human umbilical cord filtrate is mixed with a lower proportion of the micronized human umbilical cord composition. The above-mentioned packing may be repeated, as necessary.

In certain aspects, also disclosed are methods of treating an orthopedic and/or podiatric conditions/ailments. For example, in certain aspects, plantar fasciitis and/or heel ailments may be treated by injecting the mixed two-part composition disclosed herein directly into the subject's foot (subcutaneously in a portion between the ball and heel of the foot) and/or immediately adjacent to the portion of bone forming the subject's heel. This method comprises: (a) sterilely mixing the composition disclosed herein at an effective viscosity to treat a subject having orthopedic and/or podiatric conditions/ailments (e.g., plantar fasciitis); and (b) sterilely injecting the sterilely mixed composition of step (a) into and/or adjacent the area of the subject affected with orthopedic and/or podiatric conditions/ailments thereby treating the condition/ailment. In this aspect, the micronized human umbilical cord composition and the aqueous human umbilical cord filtrate are both sterile and non-immunogenic and are mixed at a ratio of 2:1 to 1:2 micronized human umbilical cord composition and the aqueous human umbilical cord filtrate to have sufficient viscosity to treat the condition/ailment. For example, when treating one's plantar fasciitis with the above method and compositions, the mixed compositions have sufficient thickness and viscosity to provide cushioning (subcutaneous cushioning) to treat and mitigate pain associated with plantar fasciitis. In particular, the high viscosity of the filtrate provides the desired cushioning effect, and the Wharton's Jelly (mucopolysaccharides and proteoglycans) in the filtrate further aid in the cushioning and protective purposes of the above-mentioned treatment(s).

In certain aspects, each individual component of the two-part clotting compositions disclosed herein may be used individually (alone) for specified purposes. For example, when using the disclosed filtrate individually, the purpose of using all filtrate (only filtrate) would be to provide the growth factors and exosomes within the filtrate without introducing a scaffolding or stromal substrate. For example, if one were to use the filtrate to provide cushioning substance to a degenerative heel pad or mix with commercially available bone particulate for application to a tooth socket. As another example and when using the disclosed micronized compositions individually (alone), the purpose of using all particulate would be to pack a wet wound bed or dental socket when the area is too wet to add additional filtrate, or another filtrate is desired, such as platelet rich plasma (PRP).

In certain additional aspects, the processed human umbilical cord composition disclosed herein that includes the aqueous human umbilical cord filtrate (but omits the micronized human umbilical cord composition) can be used for the treatment of Peyronie's disease by intracorporeal injection in a subject in need thereof. In particular, the treatment for reducing the size of Peyronie's disease plaque(s) in a human subject in need thereof includes (a) contacting (via intracoporeal injection) the Peyronie's disease plaque with a predetermined volume of the processed human umbilical cord composition having a predetermined concentration of, for example, endogenous hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), transthyretin, tissue inhibitor of metalloproteinase 1 (TIMP-1), aggrecan, or a combination thereof at a predetermined frequency in a subject (human subject) in need thereof to reduce plaque size. For example, the processed human umbilical cord composition disclosed herein may be used at the volumes and concentrations disclosed herein for 1 to 10 treatments (injections) with the composition to treat Peyronie's disease plaques or as determined by a physician with the treatment frequency ranging from every 2 to 6 weeks or as determined by the physician. In certain aspects, the aqueous human umbilical cord filtrate of the processed human umbilical cord composition comprises acellular Wharton's jelly, hyaluronan (HA) at a concentration ranging from 3.0×10⁶ pg/mL to 4.0×10⁸ pg/mL, more preferably from 4.5×10⁶ pg/mL to 3.15×10⁸ pg/mL, insulin growth factor binding protein-1 (IGFBP-1) at a concentration ranging from 2.0×10³ pg/mL to 6.0×10⁶ pg/mL, more preferably from 2.25×10³ pg/mL to 4.7×10⁶ pg/mL, sulfated glycosaminoglycans (sGAGs) at a concentration ranging from 2.0×10⁷ pg/mL to 3.0×10⁹ pg/mL, more preferably from 2.8×10⁷ pg/mL to 2.3×10⁹ pg/mL, interleukin-1 receptor antagonist (IL-1ra) at a concentration ranging from 2.0×10² pg/mL to 4.0×10⁴ pg/mL, more preferably from 3.75×10² pg/mL to 3.0×10⁴ pg/mL, transthyretin at a concentration ranging from 1.75×10² pg/mL to 6.0×10⁵ pg/mL, more preferably from 1.8×10² pg/mL to 5.3×10⁴ pg/mL, tissue inhibitor of metalloproteinase 1 (TIMP-1) at a concentration ranging from 3.0×10³ pg/mL to 8.0×10⁵ pg/mL, more preferably from 4.0×10³ pg/mL to 7.75×10⁵ pg/mL, aggrecan at a concentration ranging from 0 pg/mL to 8.0×10⁴ pg/mL, more preferably from 1×10¹ pg/mL to 7.5×10⁴ pg/mL, in which any endpoint within these ranges can serve as endpoints for any additional sub-range falling therein, or a combination thereof. In certain aspects, the above is repeated at predetermined frequencies to further reduce plaque size and/or to maintain plaque reduction in the subject.

The foregoing description provides embodiments of the invention by way of example only. It is envisioned that other embodiments may perform similar functions and/or achieve similar results. Any and all such equivalent embodiments and examples are within the scope of the present invention and are intended to be covered by the appended claims.

What is claimed is:

1. An intracorporeal injectable processed human umbilical cord composition for treatment of Peyronie's disease by intracorporeal injection in a subject in need thereof with an effective amount of the composition, the composition comprising:
(a) an aqueous human umbilical cord filtrate having interleukin-1 receptor antagonist (IL-1ra) present in the aqueous human umbilical cord filtrate at a concentration ranging from 2.0×10² pg/mL to 4.0×10⁴ pg/mL, tissue inhibitor of metalloproteinase 1 (TIMP-1) present in the aqueous human umbilical cord filtrate at a concentration ranging from 1.0×10⁴ pg/mL to 8.0×10⁵ pg/mL, insulin growth factor binding protein-1 (IGFBP-1) present in the aqueous human umbilical cord filtrate at a concentration ranging from 1.0×10⁴ pg/mL to 6.0×10⁶ pg/mL, and at least one of endogenous hyaluronic acid (HA) and/or hyaluronan, fibronectin, sulfated glycosaminoglycans (sGAGs), exosomes, hepatocyte growth factor (HGF), transthyretin, aggrecan, or a combination thereof therein at effective amount to reduce size of a Peyronie's disease plaque.

2. The composition according to claim 1, wherein the aqueous human umbilical cord filtrate has not been subjected to exogenous enzymatic digestion.

3. The composition according to claim 1, wherein the composition does not include particulates exceeding 100 μm in diameter.

4. The composition according to claim 3, wherein the aqueous human umbilical cord filtrate having at least one of acellular Wharton's jelly, hyaluronan (HA) at a concentration ranging from 3.0×10⁶ pg/mL to 4.0×10⁸ pg/mL, sulfated glycosaminoglycans (sGAGs) at a concentration ranging from 2.0×10⁷ pg/mL to 3.0×10⁹ pg/mL, transthyretin at a concentration ranging from 1.75×10² pg/mL to 6.0×10⁵ pg/mL, aggrecan at a concentration ranging from 0 pg/mL to 8.0×10⁴ pg/mL or a combination thereof.

5. The composition according to claim 4, wherein the aqueous human umbilical cord filtrate further comprises an isotonic solution.

6. The composition according to claim 5, wherein the isotonic solution is phosphate buffered saline.

7. The composition according to claim 6, wherein the aqueous human umbilical cord filtrate comprises particles from a human umbilical cord tissue that are less than 100 μm in diameter therein.

8. The composition according to claim 1, wherein the aqueous human umbilical cord filtrate is non-immunogenic.

9. A kit comprising a vial, ampule, or pre-loaded syringe having the processed human umbilical cord composition of claim 1 therein.

10. A method for reducing size of a Peyronie's disease plaque in a human subject in need thereof comprising:
(a) contacting the Peyronie's disease plaque with a predetermined volume of the composition of claim 1.

11. A method of making the composition of claim 1, the method comprising:
(a) providing a human umbilical cord;
(b) washing the human umbilical cord with an isotonic solution;
(c) grinding the washed human umbilical cord of step (b) thereby forming ground human umbilical cord tissue;
(d) separating the ground human umbilical cord tissue of step (c) into a solid retentate and an aqueous human umbilical cord supernatant;
optionally further processing the solid retentate of step (d) into a micronized human umbilical cord composition; and
(e) filtering the aqueous human umbilical cord supernatant thereby forming an aqueous human umbilical cord filtrate, the aqueous human umbilical cord filtrate having particles from the ground human umbilical cord tissue that are less than 100 μm in diameter therein, wherein:
none of steps (a)-(e) include introduction of exogenous enzymes resulting in exogenous enzymatic degradation/digestion, and
before step (d) contacting the ground umbilical cord tissue formed in step (c) with a filter and subsequently filtering the ground umbilical cord tissue to form the solid retentate retained on the filter and the aqueous human umbilical cord supernatant of step (d) that has passed through the filter, and
wherein interleukin-1 receptor antagonist (IL-1ra) is present in the aqueous human umbilical cord filtrate at a concentration ranging from 2.0×10² pg/mL to 4.0×10⁴ pg/mL.

12. The method of claim 11, wherein a grinding tool configured to grind and/or mince the washed human umbilical cord is used during step (c) and grinds the washed human umbilical cord at a range of 40 to 200 revolutions per minute (RPM) until the umbilical cord has been fully ground.

13. The method of claim 11, wherein the filter has a porosity ranging from 100 μm to 200 μm.

14. The method of claim 13, wherein the further processing step of the solid retentate is included and is a milling, freeze drying, or dehydration process that forms the micronized human umbilical cord composition having particle sizes ranging from greater than 1 μm to less than 300 μm, with the particles being polydisperse.

15. The method of claim 14, wherein the micronized human umbilical cord composition is a dried micronized human umbilical cord tissue having a particle diameter size ranging from greater than 1 μm to less than 300 μm and comprising collagen, fibronectin, IGFBP-1 at a concentration ranging from 1500 pg/mL to ˜9000 pg/mL, sGAGs at a concentration ranging from 0.1×10⁶ pg/ml to 3.0×10⁷ pg/mL, exosomes at a concentration ranging from 1.5×10⁹ particles/mL to 4.0×10⁹ and having a particle size ranging from 50 nm to 200 nm, or any combination thereof.

16. The method of claim 15, wherein the aqueous human umbilical cord filtrate comprises at least one of Wharton's jelly, hyaluronan (HA) at a concentration ranging from 3.0×10⁶ pg/mL to 4.0×10⁸ pg/mL, sulfated glycosaminoglycans (sGAGs) at a concentration ranging from 2.0×10⁷ pg/mL to 3.0×10⁹ pg/mL, transthyretin at a concentration ranging from 1.75×10² pg/mL to 6.0×10⁵ pg/mL, aggrecan at a concentration ranging from 0 pg/mL to 8.0×10⁴ pg/mL or a combination thereof.

17. The method of claim 11, wherein both the micronized human umbilical cord composition and the aqueous human umbilical cord filtrate are sterile.

18. The method of claim 11, wherein both the micronized human umbilical cord composition and the aqueous human umbilical cord filtrate are non-immunogenic.

19. The method of claim 11, further comprising:
tissue inhibitor of metalloproteinase 1 (TIMP-1) at a concentration ranging from 1.0×10⁴ pg/mL to 8.0×10⁵ pg/mL, and
insulin growth factor binding protein-1 (IGFBP-1) at a concentration ranging from 6.0×10³ pg/mL to 6.0×10⁶ pg/mL.
